(19) United States
(12) Patent Application Publication
(10) Pub. No.: US 2007/0104807 A1
(43) Pub. Date: May 10, 2007

(54) Compositions and methods for weight-loss and weight-loss maintenance in daytime and nighttime formulation

(76) Inventors: Paul T. Gardiner, Mississauga (CA); Marvin A. Heuer, Mississauga (CA)

Correspondence Address: KENYON & KENYON LLP, ONE BROADWAY, NEW YORK, NY, 10004, US

(21) Appl. No.: 11/416,847

(22) Filed: May 2, 2006

Related U.S. Application Data
(60) Provisional application No. 60/735,040, filed on Nov. 8, 2005.

(51) Int. Cl. A61K 36/82; A61K 36/28

(52) U.S. Cl. 424/729; 424/764; 424/774; 424/776; 424/777

(57) ABSTRACT

Compositions and methods for administering a diet supplement to humans are provided for the enhancement of weight loss, suppression of appetite, prevention of muscle protein catabolism, and providing antioxidants in either a daytime or nighttime formulation. Said diet supplement is comprised of at least a source of Catechins and at least 3% Corosolic Acid. The diet supplement is provided in either daytime or nighttime formulations.

### RELATED APPLICATIONS

The application is related to and claims benefit of priority to Applicant's co-pending U.S. Provisional Patent Application Ser. No. 60/735,040 entitled “Compositions and Methods for Weight-loss and Weight-loss Maintenance,” filed Nov. 22, 2005, the disclosure of which is hereby fully incorporated by reference.

### FIELD OF THE INVENTION

The present invention relates to a nutritional composition for enhancing weight loss via GLUT4 stimulation and subsequent glycogenolysis. Moreover, the present invention may serve to enhance weight loss by suppressing appetite, preventing muscle protein catabolism, providing antioxidants and/or inducing relaxation in a formulation that may be taken, e.g., immediately prior to sleep. A second embodiment may be taken upon waking from sleep, e.g., in the morning.

### SUMMARY OF THE INVENTION

The present invention provides for a composition for enhancing weight loss, suppressing appetite, preventing muscle protein catabolism, providing antioxidants and/or inducing relaxation. The composition of the present invention comprises at least a source of Catechins and at least 3% Corosolic Acid. The present invention may provide a composition and method that reduces fat body mass, and increases weight loss, leading to a desired body composition, while inducing relaxation and providing antioxidants in a stimulant-free formula. For example, the composition and method may allow a person's body to oxidize more stored body fat than they would otherwise burn, which in turn reduces body fat mass and leads to a change in body composition, ultimately leading to fat loss.

Additionally, the present invention may provide for a composition for enhancing weight loss, suppressing appetite, and preventing muscle protein catabolism. According to an embodiment of the invention, there is provided a composition and method that reduces fat body mass, and increases weight loss, leading to a desired body composition in a rapid-release technology that may be taken upon waking in the morning. For example, the composition and method may allow a person's body to oxidize more stored body fat then they would otherwise burn, which in turn reduced body fat mass and leads to changes in body composition, ultimately leading to fat loss.

### DETAILED DESCRIPTION OF THE INVENTION

The present invention, according to an embodiment thereof, is directed towards a nutritional composition for enhancing weight loss, suppressing appetite, preventing muscle protein catabolism, providing antioxidants and/or inducing relaxation. The composition may be a diet supplement. According to an embodiment of the present invention, there is provided a composition that reduces fat body mass and increases weight loss. For example, the composition and method may allow a person's body to oxidize more stored body fat than they would otherwise burn, which in turn reduces body fat, ultimately leading to fat loss.

Furthermore, in addition or alternatively, according to an embodiment of the present invention, there is provided a composition that improves or induces relaxation. For example, the present embodiment, taken as a nighttime supplement, may induce relaxation, while suppressing appetite and inducing weight loss via a non-thermogenic, glyogenolytic process.

The present invention, according to a second embodiment thereof, is directed towards a diet supplement for enhancing weight loss, suppressing appetite, and/or preventing muscle protein catabolism. According to the second embodiment of the present invention, there is provided a composition that reduces fat body mass and increases weight loss that may be taken in the morning upon waking from sleep. For example, the composition and method may allow a person's body to oxidize more stored body fat then they would otherwise burn, which in turn reduces total body fat, ultimately leading to fat loss.

In a first embodiment, the present invention may include the use of a combination, wherein the combination includes one or more of, without being limited to, Banaba Leaf Extract (containing 1%, 3%, or higher concentrations of Corosolic Acid), Caffeine-free Green Tea Leaf Extract, N-olyl-phosphatidyl ethanolamine, Caffeine-free White Tea Dry Leaf Extract, Caffeine-free Oolong Tea Dry Leaf Extract, Chamomile, and Passionflower. The supplement dosage may be consumed in any form, e.g., a capsule, a tablet, a caplet or as a dietary gel. In an embodiment, the dosage is provided in a soft gelatin capsule.

In the second embodiment, the present invention may include the use of a combination, wherein the combination includes one or more of, without being limited to, Green Tea Leaf Extract, Anhydrous Caffeine, Banaba Leaf Extract (containing 1%, 3%, or higher concentrations of Corosolic Acid), Black Tea Leaf Extract, White Tea Leaf Extract, Oolong Tea Leaf Extract, Rooibos Tea Powder, Vinpocetine, Coleus Forskholli Extract, Theobroma Cacao Extract, and Evodia Rutaecarpa Fruit Extract. The dosage may be consumed in any form, e.g., a capsule, a tablet, or as dietary gel. In an embodiment, the dosage is provided in a rapid-release capsule.

In the third embodiment, the present invention may include the use of a combination, wherein the combination includes one or more of, without being limited to, Banaba Leaf Extract (containing 1%, 3%, or higher concentrations of Corosolic Acid), Green Tea Leaf Extract, White Tea Leaf Extract, and Oolong Tea Leaf Extract. The dosage may be consumed in any form, e.g., a capsule, a tablet, or as dietary gel. In an embodiment, the dosage is provided in a soft-gelatin capsule.

Furthermore, the dosage form of the diet supplement in accordance with these embodiments may be provided in accordance with customary processing techniques for herbal and/or dietary supplements in any of the forms mentioned above.

In an embodiment of the present invention, a diet supplement may include Banaba Leaf Extract (containing 1%, 3%, or higher concentrations of Corosolic Acid), Caffeine-free Green Tea Leaf Extract, N-olyl-phosphatidyl ethanolamine, Caffeine-free White Tea Dry Leaf Extract, Caffeine-free Oolong Tea Dry Leaf Extract, Chamomile, Passionflower, Soybean oil, gelatin, glycerin, Yellow Beeswax, Purified Water, Lecithin, Titanium Dioxide, FD&C Blue #1, maltodextrin, shellac glaze, n-butyl alcohol, isopropyl alcohol, propylene glycol, and ammonium hydroxide, such as set forth in greater detail in Example 1. This diet supplement may be provided for increasing a person's natural adipose lipolytic metabolism via a non-thermogenic, glycogenolytic process.

In a second embodiment, the present invention may include Green Tea Leaf Extract, Anhydrous Caffeine, Banaba Leaf Extract (containing 1%, 3%, or higher concentrations of Corosolic Acid), Black Tea Leaf Extract, White Tea Leaf Extract, Oolong Tea Leaf Extract, Rooibos Tea Powder, Vinpocetine, Coleus Forskholli Extract, Theobroma Cacao Extract, and Evodia Rutaecarpa Fruit Extract, Microcrystalline Cellulose, Croscarmellose Sodium, Vegetable Stearine, Magnesium Strearate, Silica, Hydroxypropylcellulose, Polyvinyl Alcohol, Polysorbate Glycol, FD&C Red #40 Aluminum Lake, Talc, Titanium Dioxide, Polysorbate 80, Shellac Glaze, Isopropyl Alcohol, Propylene Glycol, Ammonium Hydroxide, Acesulfame Potassium, Maltodextrin, Sodium Glucolate, and Sodium Chloride, such as set forth in greater detail in Example 2. This diet supplement may be provided for increasing a person's natural adipose lipolytic metabolism, leading to fat loss in an accelerated weight loss formula.

In a third embodiment, the present invention may include Banaba Leaf Extract (containing 1%, 3%, or higher concentrations of Corosolic Acid), Green Tea Leaf Extract, White Tea Leaf Extract, Oolong Tea Leaf Extract, Safflower Oil, Fish Gelatin, Glycerin, Yellow Beeswax, Purified Water, Titanium Dioxide, and Cochineal Extract, such as set forth in greater detail in Example 3. This diet supplement may be provided for increasing a person's natural adipose lipolytic metabolism, leading to fat loss in an accelerated weight loss formula.

The present invention, in various stimulant-free embodiments, may be employed in a nighttime formulation for increasing a person's natural adipose metabolism via a non-thermogenic, glycogenolytic, thus leading to a desired body fat composition. Other embodiments of the present invention, that may be administered in a daytime formulation, may be employed for increasing a person's natural adipose metabolism, leading to accelerated weight loss. The compositions of the present invention may be of particular interest to those seeking to lose fat body mass and increase weight loss. The amount of the composition administered to the person may vary depending upon, e.g., the desired effect, body mass, the individual characteristics of the person, and other such factors. For example, in various embodiments, the compositions of the present invention may be administered to the diet of the person on a daily basis in nighttime formulation immediately prior to a person going to sleep, or alternatively, in a daytime formulation administered in the morning immediately after waking from sleep.

In the present invention, the insulin-responsive Glute 4 Glucose transport receptor (GLUT4) is stimulated. Studies have shown that Banaba Leaf Extract components are able to stimulate GLUT4 translocation and adipocyte differentiation inhibition in certain cells types (Liu, X., Kim, J. K., Li, Y., Li, J., Liu, F., Chen, X., “Tannic acid stimulates glucose transport and inhibits adipocyte differentiation in 3T3-L1 cells”, J. Nutr. 2005 February; 135(2):165-71). Moreover, components of Banaba Leaf Extract have also been shown to be able to reduce blood glucose via GLUT4 stimulation and translocation (Miura, T., Itoh. Y., Kaneko, T., Ueda, N., Ishida, T., Fukushima, M., Matsuyama, F., Seino, Y., “Corosolic acid induces GLUT4 translocation in genetically type 2 diabetic mice,” Biol. Pharm. Bull., 2004 July; 27(7):1103-5). Therefore, it follows that an increase in GLUT4 receptor population is accompanied by an increase in glucose being transported from the blood into brown and white adipocytes as well as skeletal muscle (Groff, J. L., Gropper, S. S., “Advanced Nutrition and Human Metabolism,” 3^{rd} Edition. Wadsworth Thomson Learning, Scarborough, Ontario. 1999). The increased amount of glucose being transported from the blood into cells leads to a decrease in blood-glucose levels which stimulates the release of pancreatic glucagon (Groff, J. L., Gropper, S. S., “Advanced Nutrition and Human Metabolism,” 3^{rd} Edition, Wadsworth Thomson Learning, Scarborough, Ontario. 1999). Glucagon then stimulates the release of fatty acids into the blood via lipolysis which are converted into glucose in the liver to restore the blood-glucose to its homeostatic level. Therefore, when the fat cells are catabolized to produce glucose, a loss in weight is observed. The present embodiments of the composition may reduce body fat mass and lead to a change in body composition via this mechanism, in part or in whole, or in combination with other mentioned mechanisms.

Moreover, in a randomized double blind clinical trial, no great increase in resting metabolic rate or in oxygen consumption was observed. This suggests that the present invention acts via a non-thermogenic mechanism. Further evidence to this end is supported by the fact that no symptoms of feeling hot, diaphoresis, or an increased temperature were reported (Judy, W. V., “A randomized double blind placebo controlled clinical trial evaluating the effects of Glucotrim® containing 3% corosolic acid on weight loss, lean and fat body mass, metabolic rate and blood sugar,” SGTI protocol WLGT 0.03-2003. 2003) during a clinical trial employing an active ingredient of the composition.

U.S. Pat. No. 6,784,206 discloses a method of manufacture of a soft-gel capsule comprising 1% Corosolic acid, wherein the Corosolic acid is absorbed into the intestinal tract of a human in order to sustain weight loss management and maintain blood sugar levels. Moreover, this patent purports to aim to improve high blood sugar levels in subjects suffering from non-insulin dependant diabetes mellitus.

The present invention, according to various embodiments, may also protect against oxidative stress through the use of antioxidants. Catechins have been shown to exhibit properties of antioxidants and thus protect against free radical damage (Ueda, J., Saito, N., Shimazu, Y., Ozawa, T., “A comparison of scavenging abilities of antioxidants against hydroxyl radicals,” Arch. Biochem. Biophys., 1996, Sep. 15; 333(2):377-84). Fatty acids are transported into the mitochondria via a covalent linkage to L-carnitine (Groff, J. L., Gropper, S. S., “Advanced Nutrition and Human Metabolism,” 3^{rd} Edition, Wadsworth Thomson Learning, Scarborough, Ontario, 1999). The oxidation of fatty acids is localized within tissues to the mitochondria, which are susceptible to oxidative stress and membrane damage. Free radicals can be formed as part of the electron transport chain which can lead to oxidative stress on the mitochondrial membrane (Groff, J. L., Gropper, S. S., “Advanced Nutrition and Human Metabolism,” 3^{rd} Edition, Wadsworth Thomson Learning, Scarborough, Ontario, 1999) leading to cellular death. Catechins scavenge reactive oxygen species and act as antioxidants, thus protecting against free radical damage (Ueda, J., Saito, N., Shimazu, Y., Ozawa, T., “A comparison of scavenging abilities of antioxidants against hydroxyl radicals,” Arch. Biochem. Biophys., 1996 Sep. 15; 333(2):377-84). The compositions of the present invention, according to various embodiments thereof, may afford protection against organelle oxidative stress via this mechanism, in part or in whole, or in combination with other mentioned mechanisms.

Moreover, epigallocatechin-3-gallate (EGCG), but not related catechins, have been experimentally shown to significantly reduced food intake and thus lead to a reduced body weight (Kao, Y. H., Hiipakka, R. A., Liao, S. “Modulation of endocrine systems and food intake by green tea epigallocatechin gallate.” Endocrinology, 2000 March; 141(3):980-7). Further to this, Catechins have also been shown to suppress intracellular lipid accumulation. The same study also suggests that catechins increase weight loss through the suppression of adipocyte differentiation (Furuyashiki, T., Nagayasu, H., Aoki, Y., Bessho, H., Hashimoto, T., Kanazawa, K., Ashida, H., “Tea catechin suppresses adipocyte differentiation accompanied by down-regulation of PPARgamma2 and C/EBPalpha in 3T3-L1 cells.” Biosci. Biotechnol. Biochem. 2004 November; 68(11):2353-9). The compositions of the present invention, according to various embodiments, may reduce body fat mass and to a change in body composition via this mechanism, in part or in whole, or in combination with other mentioned mechanisms.

The present invention, according to an embodiment thereof, may additionally acts as an appetite suppressant. Peroxisome-proliferator-activated receptors-α (PPAR-α) are found in the intestinal tract. When activated, these receptors are responsible for signaling to the hypothalamus to decrease appetite leading to a reduction in body weight (Fu, J., Gaetani, S., Oveisi, F., Lo Verme, J., Serrano, A., Rodriguez De Fonseca, F., Rosengarth, A., Luecke, H., Di Giacomo, B., Tarzia, G., Piomelli, D., “Oleylethanolamide regulates feeding and body weight through activation of the nuclear receptor PPAR-alpha.” Nature, 2003, Sep. 4; 425(6953):90-3). The compositions of the present invention, according to various embodiments, may reduce body fat mass and lead to a change in body composition via this mechanism, in part or in whole, or in combination with other mentioned mechanisms.

Furthermore, the present invention, according to various embodiments thereof, may also include compositions which initiate the transcription of proteins involved in lipid metabolism as well as repressing inducible nitric oxide synthase, an enzyme that may contribute to feeding stimulation (Fu, J., Gaetani, S., Oveisi, F., Lo Verme, J., Serrano, A., Rodriguez De Fonseca, F., Rosengarth, A., Luecke, H., Di Giacomo, B., Tarzia, G., Piomelli, D., “Oleylethanolamide regulates feeding and body weight through activation of the nuclear receptor PPAR-alpha,” Nature, 2003 Sep. 4; 425(6953):90-3). The compositions of the present invention, according to various embodiments, may reduce body fat mass and lead to a change in body composition via this mechanism, in part or in whole, or in combination with other mentioned mechanisms.

Moreover, the present invention, according to various embodiments thereof, may also include compositions which may be effective in inducing relaxation and possessing anxiolytic properties via binding to the benzodiazepine binding site in the gamma-aminobutyric acid receptor type A (GABA(A)) (Fernandez, S. P., Wasowski, C., Paladini, A. C., Marder, M., “Synergistic interaction between hesperidin, a natural flavonoid, and diazepam,” Eur. J. Pharmacol., 2005 Apr. 11; 512(2-3):189-98). The action of this ligand leads to sedation and a reduction in locomotor activity (Avallone, R., Zanoli, P., Puia, G., Kleinschnitz, M., Schreier, P., Baraldi, M., “Pharmacological profile of apigenin, a flavonoid isolated from Matricaria chamomilla,” Biochem. Pharmacol., 2000 Jun. 1; 59(11):1387-94) without impairing memory or motor skills (Salgueiro, J. B., Ardenghi, P., Dias, M., et al., “Anxiolytic natural and synthetic flavonoid ligands of the central benzodiazepine receptor have no effect on memory tasks in rats.” Pharmacol. Biochem. Behav., 1997; 58(4):887-91). The compositions of the present invention, according to various embodiments, may act as a sedative leading to the induction of relaxation. The compositions of the present invention, according to various embodiments, may reduce body fat mass by inducing sleep so as to prevent food intake and thus to prevent increased levels of glucose being stored as fat or functional stress which again may increase blood-glucose levels.

The present invention, according to various embodiments thereof, provides a method which includes consuming a composition, wherein the method enhances weight loss, suppressing appetite, preventing muscle protein catabolism, providing antioxidants and/or inducing relaxation. According to another embodiment, there is provided a method which includes consuming a composition, wherein the method enhances weight loss, suppresses appetite, and/or prevents muscle protein catabolism. The method of the present invention may include the step of consuming a composition in the form of a nighttime diet supplement, whereas the method of another embodiment of the present invention may include the step of consuming a composition in the form of a daytime diet supplement. According to various embodiments of the present invention, the method includes the step of consuming a composition that reduces body fat mass, leading to a change in body composition.

Consuming components, such as those in a composition of the present invention, while on a standardized diet, subjects lost on average 45% more weight than a placebo group (Judy, W. V., “A randomized double blind placebo controlled clinical trial evaluating the effects of Glucotrim® containing 3% corosolic acid on weight loss, lean and fat body mass, metabolic rate and blood sugar,” SGTI protocol WLGT 0.03-2003, 2003) over the course of a clinical trial employing an active ingredient of the composition. Additionally, there was a corresponding reduction in the circumference of body segments comprised of waist, hip, upper arm and thigh. Subjects in the treatment group showed a 35% increase in total centimeters lost relative to control groups. For example, the consumption of the composition in accordance with the method of the present invention, may allow a person's body to burn more or otherwise lose stored body fat than they would otherwise burn or lose, leading to a reduction in weight as well as reduction in body volume.

In one embodiment of the present invention, the method includes the prior-to-sleep consumption of a combination, wherein the combination includes one or more of, without being limited to, Banaba Leaf Extract (containing 1%, 3%, or higher concentrations of Corosolic Acid), Caffeine-free Green Tea Leaf Extract, N-olyl-phosphatidyl ethanolamine, Caffeine-free White Tea Dry Leaf Extract, Caffeine-free Oolong Tea Dry Leaf Extract, Chamomile, and Passionflower.

In a second embodiment of the present invention, the method includes the upon-waking-from-sleep consumption of a combination, wherein the combination includes one or more of, without being limited to, Green Tea Leaf Extract, Anhydrous Caffeine, Banaba Leaf Extract (containing 1%, 3%, or higher concentrations of Corosolic Acid), Black Tea Leaf Extract, White Tea Leaf Extract, Oolong Tea Leaf Extract, Rooibos Tea Powder, Vinpocetine, Coleus Forskholli Extract, Theobroma Cacao Extract, and Evodia Rutaecarpa Fruit Extract.

In a third embodiment of the present invention, the method comprises the prior-to-sleep consumption of a combination of one or more, but not limited to Banaba Leaf Extract (containing 1%, 3%, or higher concentrations of Corosolic Acid), Green Tea Leaf Extract, White Tea Leaf Extract and Oolong Tea Leaf Extract.

The supplement compositions according to the present invention, may be utilized in methods to enhance weight loss by suppressing appetite, preventing muscle protein catabolism, providing antioxidants and inducing relaxation in a formulation designed to be taken immediately prior to sleep. Another embodiment may be taken immediately upon waking from sleep in the morning. The methods of the present invention may be of particular interest to those seeking to lose body fat mass. The method may involve a determination, and an administration, of an amount of a composition in accordance with factors such as the desired effect, the body weight and characteristics of the person and the like. For example, in various embodiments, the method includes administration of the aforementioned compositions to the diet of a person on a daily basis.

Although the following examples illustrate the practice of the present invention in three of its embodiments, the examples should not be interpreted as limiting the scope of the invention. Other embodiments of the present invention will be apparent to those skilled in the art from consideration of the specification and example.

### EXAMPLE 1

A diet supplement is provided in a soft-gel formulation comprising Leaf Extract of Lagestroemia Speciosa (0.0240 g) standardized to 3% Corosolic acid, Caffeine-free Green Tea dry leaf extract (0.0010 g) standardized to 45% EGCG, 75% Catechins, 90% Polyphenols, N-olyl-phosphatidyl ethanolamine(NOPE)/EGCG blend (0.0010 g) standardized to 23% NOPE, 20% Polyphenols, 14% EGCG, Caffeine-free White Tea dry lead extract (0.0010 g) standardized to 15% EGCG, 35% Catechins, 50% Polyphenols, Caffeine-free Oolong Tea dry leaf extract (0.0010 g) standardized to 15% EGCG, 35% Catechins, 50% Polyphenols, Chamomile (0.0010 g) standardized to 1.2% Apigenin, and Passionflower (0.0010 g) supplying 3.5% flavanoids.

### EXAMPLE 2

A diet supplement is provided utilizing a rapid release technology formulation comprising Green Tea Leaf Extract (0.2000 g) standardized to 45% EGCG, 75% Catechins, 90% Polyphenols, Anhydrous Caffeine (0.2000 g), Leaf Extract of Lagestroemia Speciosa (0.0240 g) standardized to 3% Corosolic acid, Black Tea Leaf Extract (0.0010 g) standardized to 25% EGCG, 50% Catechins, 70% Polyphenols, White Tea Leaf Extract (0.0010 g) standardized to 15% EGCG, 25% Catechins, 50% Polyphenols, Oolong Tea Leaf Extract (0.0010 g) standardized to 15% EGCG, 25% Catechins, 50% Polyphenols, Rooibos Tea Powder (0.0010 g) standardized to 20% Polyphenols, Vinpocetine (0.0010 g), Coleus Forskholli Extract (0.0010 g) standardized to 10% Forskolin, Theobroma Cacao Extract (0.0010 g) standardized to 6% Theobromine, and Evodia Rutaecarpa Fruit Extract (0.0010 g) standardized to 10% Evodiamine.

### EXAMPLE 3

A diet supplement is provided in a soft-gel formulation comprising Leaf Extract of Lagestroemia Speciosa (0.0240 g) standardized to 3% Corosolic acid, Green Tea Leaf Extract (0.0010 g) standardized to 18% EGCG, 26% Catechins, 45% Polyphenols, White Tea Leaf Extract (0.0010 g) standardized to 13% EGCG, 22% Catechins, 40% Polyphenols, Oolong Tea Leaf Extract (0.0010 g) standardized to 13% EGCG, 22% Catechins, 40% Polyphenols, Safflower Oil, Fish Gelatin, Glycerin, Yellow Beeswax, Purified Water, Titanium Dioxide, and Cochineal Extract.

What is claimed:

1. A diet supplement for promoting weight loss comprising at least 3% Corosolic acid and a source of Catechins.

2. The diet supplement of the claim 1 further, comprising Chamomile Extract.

3. The diet supplement of claim 1 further, comprising Green Tea Extract.

4. The diet supplement of claim 2, further comprising Caffeine-free White Tea Extract.

5. The diet supplement of claim 4, further comprising Caffeine-free Oolong Tea Extract.

6. The diet supplement of claim 3, further comprising White Tea Extract.

7. The diet supplement of claim 6, further comprising Oolong Tea Extract.

8. The diet supplement of claim 5, further comprising Passionflower Extract.

9. The diet supplement of claim 8, further comprising Caffeine-free Green Tea Extract.

10. The diet supplement of claim 9, further comprising a blend of N-olyl-phosphatidyl ethanolamine and Epigal locatechin-3-gallate.

11. The diet supplement of claim 7, further comprising Rooibos Tea Powder.

12. The diet supplement of claim 11, further comprising Vinpocetine.

13. The diet supplement of claim 12, further comprising Coleus Forskholli.

14. The diet supplement of claim 13, further comprising Theobroma Cacao Extract.

15. The diet supplement of claim 14, further comprising Evodia Rutaecarpa Fruit Extract.

16. The diet supplement of claim 15, further comprising Black Tea Extract.

17. A method for enhancing weight loss comprising the step of administrating a diet supplement that comprises at least 3% Corosolic acid and a source of Catechins.

18. A method for improving lean body mass comprising the step of administrating a diet supplement that comprises at least 3% Corosolic Acid and a source of Catechins.

19. A method for inducing relaxation comprising the step of administrating a diet supplement that comprises at least 3% Corosolic acid and Apigenin.

20. A method of appetite suppression comprising the step of administrating a diet supplement that comprises at least 3% Corosolic acid and a source Catechins.

21. The diet supplement of claim 16 to be taken immediately upon waking in the morning.

22. The diet supplement of claim 16 comprising a rapid-release oral mode of delivery.

23. The diet supplement of 7 comprising a soft-gelatin caplet oral mode of delivery.

24. The diet supplement of claim 9 comprising a soft-gelatin caplet oral mode of delivery.

25. The diet supplement of claim 22, comprising in daytime formulation.

26. The diet supplement of claims 23 and 24 comprising a nighttime formulation.
